(19) United States
(12) Patent Application Publication
(10) Pub. No.: US 2025/0057676 A1
(43) Pub. Date: Feb. 20, 2025

(54) SINGLE MEMBER INTRALUMINAL DEVICE AND METHOD OF FIXATION

(71) Applicant: BFKW, LLC, Grand Rapids, MI (US)

(72) Inventor: Randal S. Baker, Grand Rapids, MI (US)

(21) Appl. No.: 18/914,921

(22) Filed: Oct. 14, 2024

Related U.S. Application Data
(63) Continuation of application No. 17/436,935, filed on Sep. 7, 2021, now Pat. No. 12,138,188.
(60) Provisional application No. 62/823,292, filed on Mar. 25, 2019.
(60) Provisional application No. 62/816,629, filed on Mar. 11, 2019.

(51) Int. Cl. A61F 5/00

(52) U.S. Cl. CPC A61F 5/0076

(57) ABSTRACT

An intraluminal device and method of applying adjustable pressure to an inner portion of a lumen includes deploying the intraluminal device to the inner portion of the lumen. The intraluminal device has a body with a wall defining a surface that is configured to the inner portion of the lumen. The body is fixed to the inner portion of the lumen with an anchor system in a manner that said surface is adapted to apply at least intermittent pressure to the inner portion of the lumen. The pressure applied by the surface to the inner portion of the lumen is controlled with a control system. The control system has a controller joined with at least two spaced apart locations at the wall. The controller distorts the wall by causing warping or rolling of the wall by modifying distance between said at least two spaced apart locations.

### CROSS REFERENCE TO RELATED APPLICATIONS

The present invention is a continuation of U.S. patent application Ser. No. 17/436,935, filed Sep. 7, 2021, which is a § 371 national stage of International Application PCT/IB2020/052169, filed Mar. 11, 2020, which claims the benefit of U.S. provisional applications, Ser. No. 62/823,292, filed Mar. 25, 2019, and Ser. No. 62/816,629, filed Mar. 11, 2019, all of which are hereby incorporated herein by reference in their entireties.

### BACKGROUND OF THE INVENTION

The present invention is directed to a method and structure for resisting migration of an intraluminal device in a lumen and in particular to a single member intraluminal device that is fixed to a portion of the gastrointestinal (GI) tract. While it may be used in other portions of the GI tract, the invention is illustrated with a single member intraluminal device fixed to the cardiac portion of the stomach or adjacent the gastro-esophageal (EG) junction.

### SUMMARY OF THE INVENTION

The embodiments of the present invention are useful for adjusting pressure applied by an intraluminal device such as a bariatric device and method as disclosed in commonly assigned U.S. Pat. No. 9,198,789 and international application publication WO 2016/109346 or as a metabolic disease treatment device and method as disclosed in commonly assigned international application publication WO 2015/031077, the disclosures of which are hereby incorporated herein by reference. Other applications will be apparent to the skilled artisan. A particularly difficult problem is to make adjustable the force applied by a generally planar member, such as a cardiac member in the applications set forth above, to the inner wall of a lumen, particularly one that experiences peristalsis, such as the cardiac portion of the stomach. While other solutions are known in the art, the embodiments of the present invention allows adjustment of pressure applied by a planar member which comprises the intraluminal device.

An intraluminal device and method of applying adjustable pressure to an inner portion of a lumen, according to an aspect of the invention, includes deploying the intraluminal device to the inner portion of the lumen. The intraluminal device has a body with a wall defining a surface that is configured to the inner portion of the lumen. The body is fixed to the inner portion of the lumen with an anchor system in a manner that said surface is adapted to apply at least intermittent pressure to the inner portion of the lumen. The pressure applied by the surface to the inner portion of the lumen is controlled with a control system. The control system has a controller joined with at least two spaced apart locations at the wall. The controller distorts the wall by causing warping or rolling of the wall by modifying distance between said at least two spaced apart locations.

The controller may be mounted to the wall. The controller may be connected with at least one of the spaced apart locations with a filament and adapted to adjust length of the filament to adjust the pressure applied by the surface to the inner portion of the lumen. The anchor system may include a plurality of anchors and one said at least two spaced apart locations may be proximate to one of said anchors. The surface may be configured to the cardiac portion of the stomach. The wall may have a central opening that is configured to align with an esophageal-gastric junction of a patient and a peripheral edge. The plurality of anchors may be distributed around the central opening.

### BRIEF DESCRIPTION OF THE DRAWINGS

FIG. 1 is an elevational view of an intraluminal device according to embodiments of the invention deployed to a recipient;

FIG. 2 is an elevation view of the intraluminal device in FIG. 1;

FIG. 3 is a view of the intraluminal device in FIG. 1 taken from the direction III-III in FIG. 2;

FIG. 3A us a sectional view taken along the lines IIIa-IIIa in FIG. 3;

FIG. 4 is the same view as FIG. 3 of an alternative embodiment;

FIG. 5 is the same view as FIG. 3 of another alternative embodiment;

FIG. 6 is the same view as FIG. 3 of an embodiment of an intraluminal device capable of applying adjustable pressure to the lumen wall;

FIG. 7 is an elevation view of an intraluminal device according to an alternative embodiment of an intraluminal device;

FIG. 8 is a sectional view taken along the lines VIII-VIII in FIG. 7; and

FIG. 9 is a bottom plan view taken from the direction IX-IX in FIG. 7.

### DESCRIPTION OF THE PREFERRED EMBODIMENTS

The present invention will now be described with reference to the accompanying figures, wherein the numbered elements in the following written description correspond to like numbered elements in the figures.

Referring now to the drawings and the illustrative embodiments depicted therein, an intraluminal device 10 includes a body 12 made up of a flat pattern, such as a weave, of a filament made of nitinol wire, plastic filament, carbon filament or the like, covered with bio-compatible layers such as silicone, thereby defining a wall 14 having a surface 16 that is configured to a portion of the lumen. In the illustrated embodiment, body 12 is configured to apply pressure to the cardiac portion of the stomach and has a central opening 15 that is adapted to be aligned with the EG sphincter in order to pass food through opening 15. Body 12 has an outer edge portion 28 that is shown as circular so that surface 16 generally covers the inner wall 18 of the lumen, such as the cardiac portion of the stomach. Other shapes can be used.

An anchor system 20 which, alone or in combination with another anchoring technique, is configured to fix body 12 to inner wall 18 of the lumen. It does so in a manner that surface 16 is capable of applying at least intermittent pressure to inner wall 18 of the lumen. Anchor system 20 is made up of a plurality of anchors 25. Each anchor 25 includes a fastener 22 that is capable of at least partially retaining wall 18 with a tissue fold 24 of the lumen that is adjacent to wall 18.

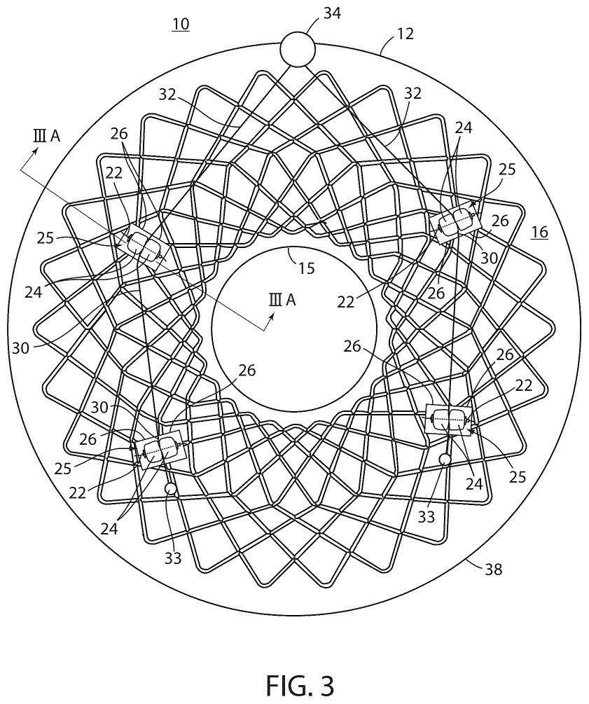

In the embodiment illustrated in FIG. 3a, each anchor 25 is made up of an opening 26 that is traversed by a crossbar 30 bisecting opening 26. With a pair of tissue folds 24 of the lumen extending into each opening 26, a fastener 22 couples the tissue folds 24 in a manner that retains crossbar 30 between the tissue folds. In the illustrated embodiment, 4 anchors are spaced from both central opening 15 and peripheral edge portion 38 of wall 14. This evenly distributes the pressure applied by surface 16 to inner wall 18 of the lumen. Of course, a greater or fewer number of anchors 25 can be distributed generally evenly about central opening 15 in wall 18. Tissue folds 24 may be formed and pulled into opening 26 and fastener 22 may be deployed according to the principles set forth in U.S. Pat. No. 10,172,608, the disclosure of which is hereby incorporated herein by reference. The first tissue fold is formed and pulled into opening 26 then retained by passing fastener 22 through that fold. The second tissue fold is then formed and pulled into opening 26 on the other side of crossbar 30 and fastened with the other tissue fold by passing the same fastener 22 through the second tissue fold. Other endoscopic fastener devices may be used such as a full-bite suturing device from Apollo Endosurgical and the like. Alternatively, a single tissue fold may be formed and pulled through opening 26 and retained in the opening by a mechanical clip that is larger in size than the opening.

The anchors 25 are adapted to be disabled in order to explant body 12 from the lumen. In the embodiment illustrated in FIG. 3, anchors 25 can be disabled by the crossbar 30 of each anchor being able to be removed from between openings 26. Crossbars 30 can be interconnected in a crossbar assembly 32 so they can be removed together and thereby defeat anchor system 20 in order to explant body 12. Crossbar assembly 32 can be removed by being attached at one end with a removable attachment 33 with wall 14 and having a grasping portion 34 at an opposite end of the crossbar assembly. Removable attachment 33 can be a suture or the like and holds crossbar assembly 32 in place during deployment in the lumen. When attachment 33 is severed, such as with an endoscopic scissor or other cutting implement, the crossbar assembly can be axially withdrawn from each anchor 25 by grasping portion 34 with an endoscopic tool and pulling on the crossbar assembly. This axially withdraws crossbar 30 from each anchor 25. Grasping portion 34 may be an enlarged area such as a bead, loop, or the like. Crossbar assembly 32 including crossbars 30, removable attachment 33 and grasping portion 34 are assembled to body 12 prior to deployment of the intraluminal device in the lumen with anchors 25 completed after deployment of the body in the lumen.

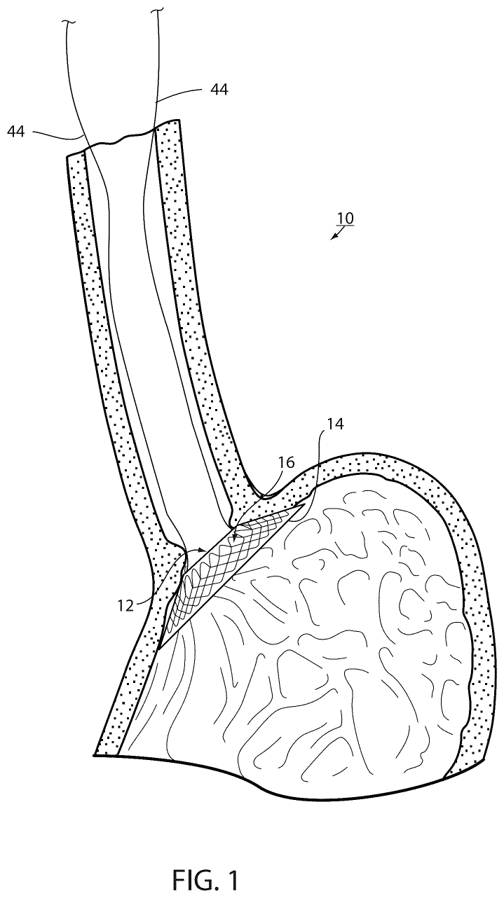

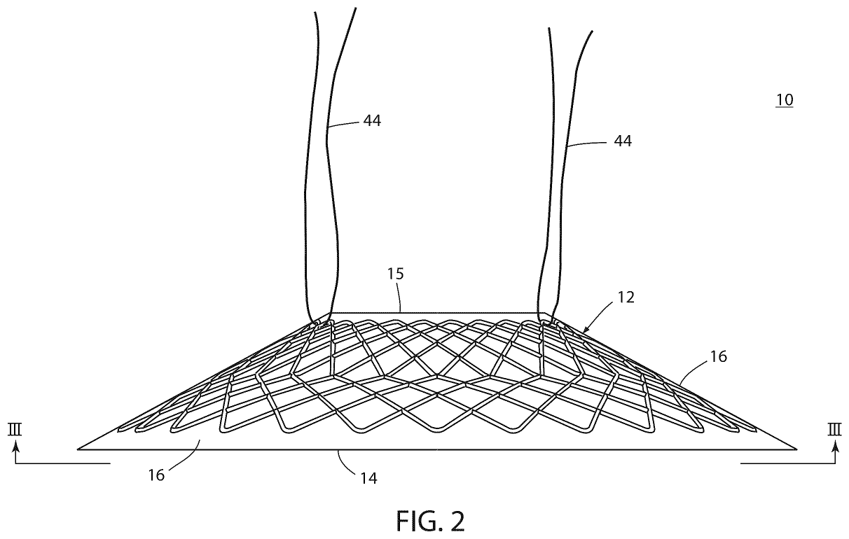

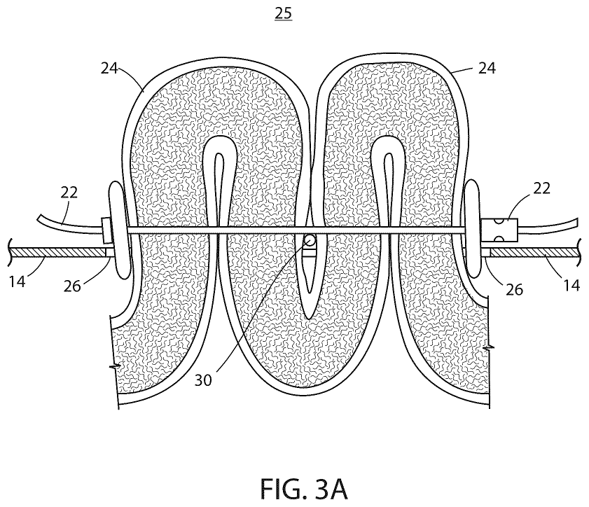

In order to deploy intraluminal device 10, body 12 is deployed to the stomach with a deployment device of the type disclosed in commonly assigned U.S. Pat. No. 9,545,326, the disclosure of which is hereby incorporated herein by reference. Central opening 15 is aligned with the esophageal gastric junction using the deployment device traversing the EG junction and opening 15. Retention filaments 44 extending from wall 14 to external the recipient, as shown in FIGS. 1 and 2, can be used to retain wall 14 to the cardiac portion of the stomach of the recipient during deployment. Retention filaments 44 are looped around an opening in wall 14 so they may be withdrawn following deployment by pulling on one filament of the loop. In the alternative embodiment illustrated in FIG. 4, intraluminal device 110 includes a body 112 anchored to the inner wall of the lumen with an anchor system 120 made up of anchors 25 with at least one opening 26 in wall 14 and an optional crossbar 30 (not shown in FIG. 4) at an edge portion of opening 26. A tissue fold 24 is positioned in opening 26 and anther tissue fold 24 outside of edge portion 28 of wall 14. If a cross bar is used with each anchor 25 they can be joined in a cross bar assembly 22 as previously described with respect to intraluminal device 10. If no cross bar is used, then the anchor can be disabled by severing fastener 22.

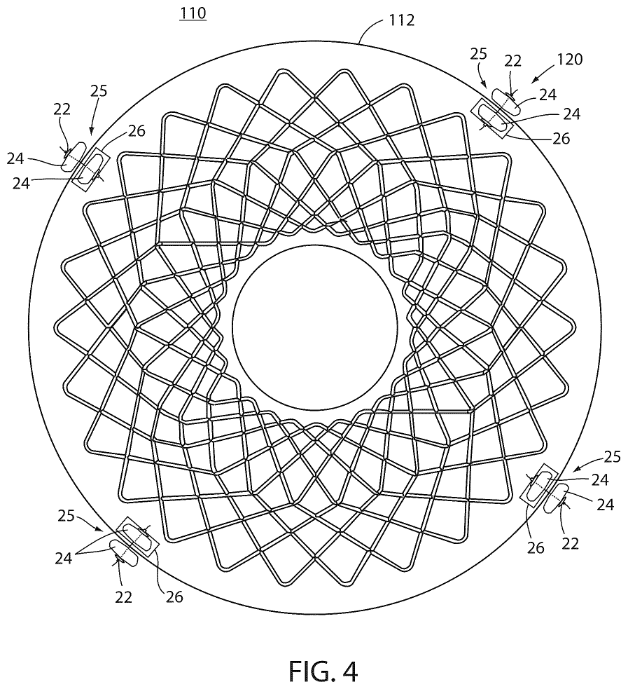

In the alternative embodiment illustrated in FIG. 5, intraluminal device 210 includes a body 212 with no opening provided in wall 14. Each anchor 225 includes a tissue fold 24 outside of edge portion 28 of wall 14 that is connected directly to wall 14 with a fastener 22 that penetrates the tissue fold 24 and the wall 14. The anchor 225 can be disabled by severing fastener 22.

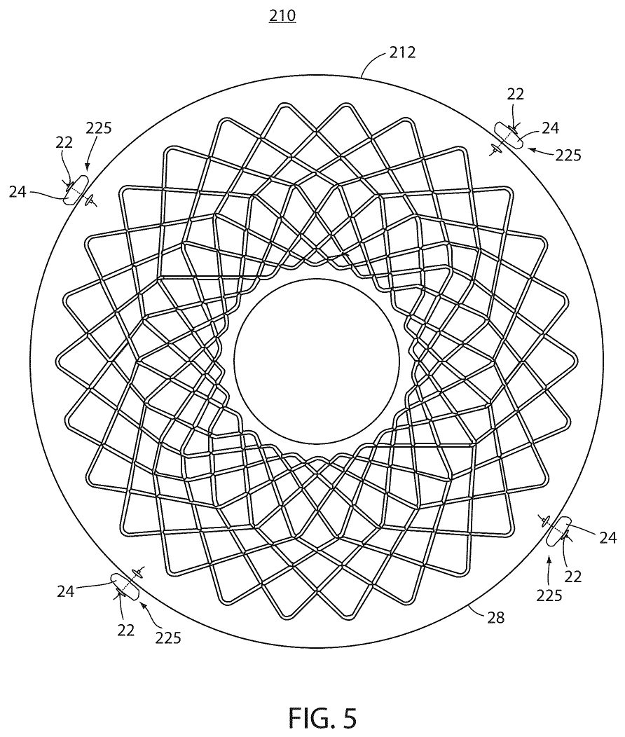

An intraluminal device 310 illustrated in FIG. 6 is capable of applying an adjustable pressure with surface 316 of a body 312 to the inner wall of the lumen. Surface 316 is moveable with one or more inner bladders 36 and a control system 38 that adjusts inflation of inner bladder(s) 36 using a controller 40 and one or more outer bladders 42 on an opposite side of wall 14. Controller 40 exchanging a fluid between bladders 36 and 42 using pumps 46 in order to move surface 316 toward and away from the inner wall of the lumen.

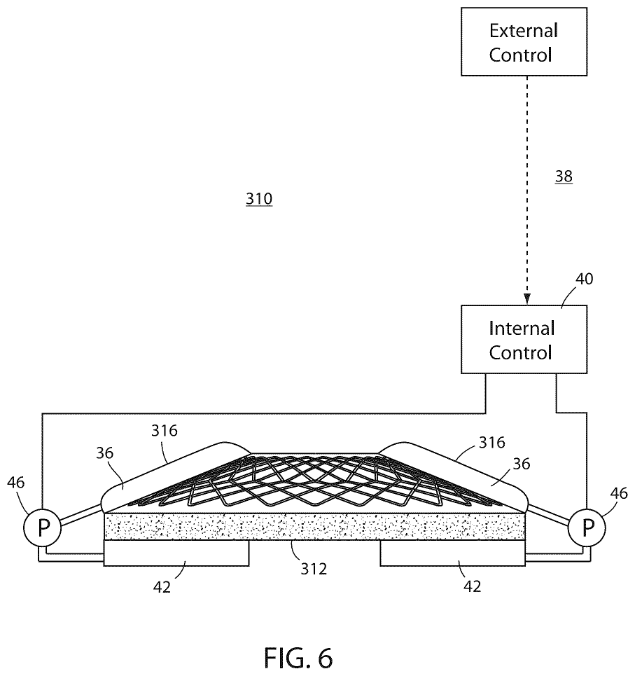

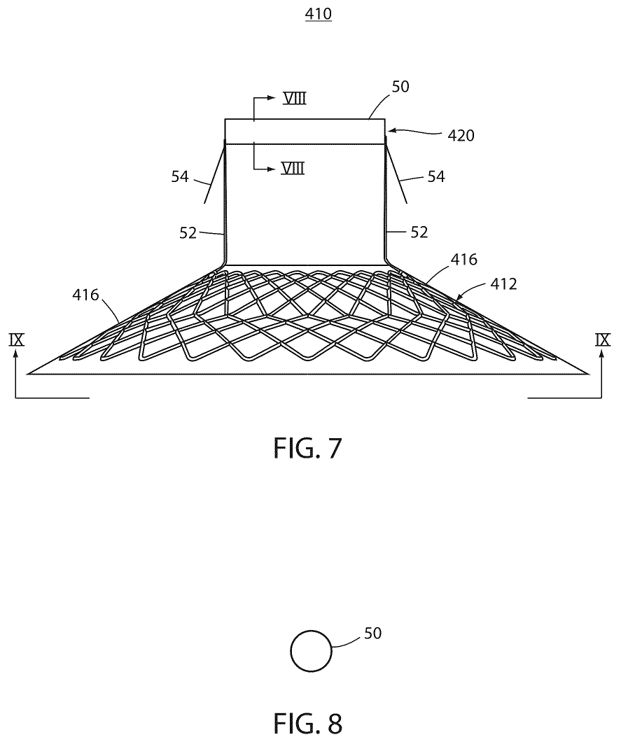

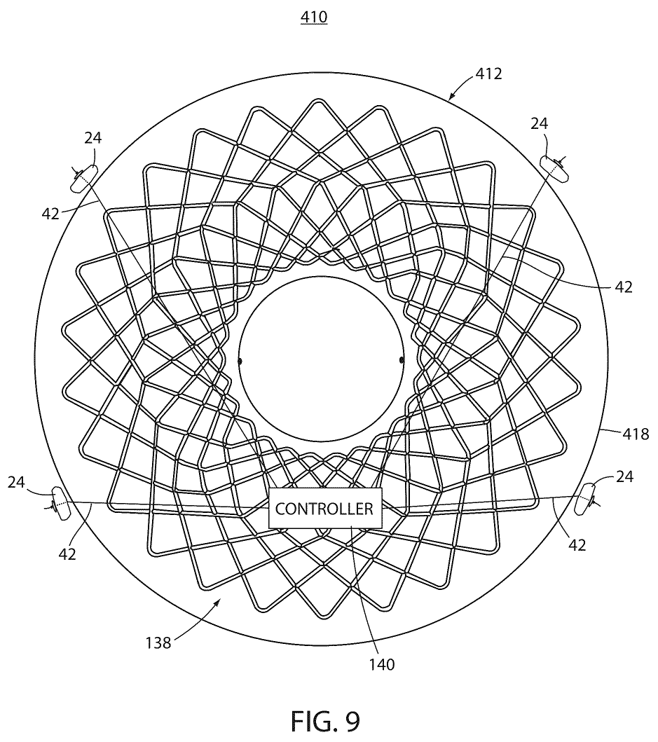

Another embodiment of an intraluminal device 410 includes a body 412 having a surface 416 that applies at least intermittent pressure to the inner wall of the lumen. As seen in FIG. 7, device 410 utilizes another body 50 and two or more connectors 52 connecting with body 412 as an additional anchor system 420 to help fix body 412 to a portion of a lumen. Body 412 is configured to the size and shape of the cardiac portion of the stomach and another body 50 is configured to the size and shape of the distal portion of the esophagus. Another body 50 may be anchored in the distal esophagus using various techniques disclosed in the art by the present inventor. In the illustrated embodiment, anchor system 420 utilizes at least one distally directed tine 54 that at least partially penetrates the esophageal-gastric junction in the manner disclosed in commonly assigned patent application U.S. Ser. No. 62/823,259, entitled INTRALUMINAL DEVICE AND METHOD WITH ANTI-MIGRATION, filed Mar. 25, 2019 (attorney docket BAK04 P128) the disclosure of which is hereby incorporated herein by reference. Connectors 52 cause body 412 to apply at least an intermittent force to the cardiac portion of the stomach with surface 416. Another body 50 may have a generally curvilinear cross section as illustrated in FIG. 8, in order to minimize potential for irritating the esophagus, or some other shape. Alternatively, intraluminal device 410 may utilize another body 50 and connectors 52 just to provide an alignment of its center opening 15 with the esophagus. This ensures that food and other intraluminal content pass body 412 without any interference. Also, body 50 and connectors 52 retain the axial orientation of body 412. In such alternative embodiment, tines 54 are absent as no additional fixation is provided by body 50 and connectors 52.

With intraluminal device 410 anchored against the inner wall of the cardiac portion of the stomach, a plurality of tissue folds 24 are a part of control system 138. Control system 138 includes a controller 140 that is attached to the inner surface of body 412 and is connected with tissue folds 24 with filaments 42. Controller 140 adjusts pressure applied by surface 416 to the cardiac portion of the stomach by adjusting the length of filaments 42. As the opposite end of filaments 42 are joined with the inner wall of the cardiac portion of the stomach with tissue folds 24, the shortening of filaments 42 by controller 140 causes distortion on wall 412 such as by warping or rolling which causes the pressure applied by surface 416 to change. Increasing the length of filaments 24 has the opposite affect. Thus in intraluminal device 410 tissue folds 24 are primarily concerned with control of the amount of pressure applied to the cardiac portion of the stomach than to anchoring of the device body. Other applications of tissue folds will be apparent to the skilled artisan in view of the disclosures herein.

Embodiments of the invention may be used to provide fixation for use with the technology disclosed in commonly assigned U.S. Pat. No. 9,055,998 the disclosure of which is hereby incorporated herein by reference. Several of the embodiments disclosed in the '998 patent include single member intraluminal devices which could make use of the fixation techniques provided herein to provide sole or additional fixation of the intraluminal devices in the lumen of the recipient.

While the foregoing description describes several embodiments of the present invention, it will be understood by those skilled in the art that variations and modifications to these embodiments may be made without departing from the spirit and scope of the invention, as defined in the claims below. The present invention encompasses all combinations of various embodiments or aspects of the invention described herein. It is understood that any and all embodiments of the present invention may be taken in conjunction with any other embodiment to describe additional embodiments of the present invention. Furthermore, any elements of an embodiment may be combined with any and all other elements of any of the embodiments to describe additional embodiments.

The embodiments of the invention in which an exclusive property or privilege is claimed are defined as follows:

1. An intraluminal device, comprising:
a body having a wall defining a surface, said surface configured to an inner portion of a lumen;
an anchor system, said anchor system configured to fix said body with said portion of the lumen in a manner that said surface is adapted to apply at least intermittent pressure to the inner portion of the lumen, and
a control system, said control system comprising a controller joined with at least two spaced apart locations at the wall, said controller adapted to distort said wall by modifying distance between said at least two spaced apart locations to cause warping or rolling of said wall in order to adjust the pressure applied by said surface to the inner portion of the lumen.

2. The device as claimed in claim 1 wherein said controller is mounted to said wall.

3. The device as claimed in claim 1 wherein said controller is connected with at least one of the spaced apart locations with a filament, said controller adapted to adjust length of the filament to adjust the pressure applied by the surface to the inner portion of the lumen.

4. The device as claimed in claim 3 wherein said anchor system comprises a plurality of anchors and wherein one said at least two spaced apart locations is at one of said anchors.

5. The device as claimed in claim 4 wherein said surface is configured to the cardiac portion of the stomach.

6. The device as claimed in claim 1 wherein said wall has a central opening that is configured to align with an esophageal-gastric junction of a patient and a peripheral edge and wherein said plurality of anchors are distributed around said central opening.

7. The device as claimed in claim 1 wherein said surface is configured to the cardiac portion of the stomach.

8. The device as claimed in claim 7 wherein said wall has a central opening that is configured to align with an esophageal-gastric junction of a patient and a peripheral edge and wherein said anchor system comprises a plurality of anchors distributed around said central opening.

9. A method of applying adjustable pressure to an inner portion of a lumen, comprising:
deploying an intraluminal device to the inner portion of the lumen, said intraluminal device having a body with a wall defining a surface, said surface configured to the inner portion of a lumen;
fixing the body to the inner portion of the lumen with an anchor system in a manner that said surface is adapted to apply at least intermittent pressure to the inner portion of the lumen, and
controlling the pressure applied by said surface to the inner portion of the lumen with a control system, said control system having a controller joined with at least two spaced apart locations at the wall, said controller distorting said wall by causing warping or rolling of said wall by modifying distance between said at least two spaced apart locations.

10. The method as claimed in claim 9 wherein said controller is mounted to said wall.

11. The method as claimed in claim 9 wherein said controller is connected with at least one of the spaced apart locations with a filament and adapted to adjust length of the filament to adjust the pressure applied by the surface to the inner portion of the lumen.

12. The method as claimed in claim 11 wherein said anchor system comprises a plurality of anchors and wherein one said at least two spaced apart locations is proximate to one of said anchors.

13. The method as claimed in claim 12 wherein said surface is configured to the cardiac portion of the stomach.

14. The method as claimed in claim 13 wherein said wall has a central opening that is configured to align with an esophageal-gastric junction of a patient and a peripheral edge and wherein said plurality of anchors are distributed around said central opening.

15. The method as claimed in claim 9 wherein said surface is configured to the cardiac portion of the stomach.

16. The method as claimed in claim 15 wherein said wall has a central opening that is configured to align with an esophageal-gastric junction of a patient and a peripheral edge and wherein said anchor system comprises a plurality of anchors distributed around said central opening.
